(19) United States
(12) Patent Application Publication
(10) Pub. No.: US 2007/0048749 A1
(43) Pub. Date: Mar. 1, 2007

(54) Method for survival prediction in gastric cancer patients after surgical operation using gene expression profiles and application thereof

(76) Inventors: Chiung-Nien Chen, Tainan Hsien (TW); Jen-Jen Lin, Taipei (TW); Fon-Jou Hsieh, Taipei (TW); King-Jen Chang, Taipei (TW)

Correspondence Address: BIRCH STEWART KOLASCH & BIRCH, PO BOX 747, FALLS CHURCH, VA, 22040-0747, US

(21) Appl. No.: 11/284,845

(22) Filed: Nov. 23, 2005

(30) Foreign Application Priority Data
Aug. 30, 2005 (TW) 094129740

(51) Int. Cl. C12Q 1/68; G06F 19/00

(52) U.S. Cl. 435/6; 702/20

(57) ABSTRACT

Disclosed is a method for survival prediction in gastric cancer patents after surgical operation, which uses a survival prediction model determined by known statistical method and gene expression microarray profiles. The survival prediction model is established by selecting special genes expressing significantly differential from pairs of cancerous and noncancerous tissue samples from patients with known survival conditions after surgical operation, confirming the concordance of RT-PCR analysis with the microarray gene expression profile, identifying most specific genes among the special genes using a statistical method, and determining the survival prediction model based on training set samples. The method of the present invention can be applied in gastric cancer patients to predict survival conditions after surgical operation and to provide a strategy for succeeding treatment and a reference for adjuvant chemotherapy.

### BACKGROUND OF THE INVENTION

1. Field of the Invention

The present invention relates to a method for survival prediction after surgical operation of cancer, especially relates to a method using microarray gene expression profile and reverse transcriptase chain reaction to predict survival of gastric cancer patients after surgical operation.

2. The Prior Arts

Gastric cancer is one of the most frequent cancers in the world, which ranks the fourth most common in Taiwan. Endoscopic screening is clinically used at present to diagnose early stage diseases. However, there are patients with advanced tumors at the time of diagnosis. According to previous reports, patients with stage I disease have a good prognosis, and those with stage IV disease show a very poor prognosis. Bewilderedly, the prognosis varies widely in patients with stage II or III disease for as of yet undetermined biologic reasons.

Some of the research papers indicated that the traditional clinicopathological factors and several interesting molecules including cell cycle regulation factors such as p27 or cyclin E, cell adhesion molecules such as E-cadherin, angiogenic factors such as vascular endothelial growth factor and placenta growth factor, oncogenes such as c-erbB2 and c-myc, tumor suppressor genes such as p53, have been correlated with the prognosis of gastric cancer patients. However, inconsistencies among different studies were found. These parameters provided limited information about prognosis of individual patients because of complex biology behind the disease. The cellular and molecular heterogeneity of gastric cancers and the large number of genes potentially involved in the multi-step process of gastric cancer pathogenesis emphasize the importance of studying multiple genetic alterations in concert.

Recent advances in the DNA microarray technique that can investigate gene expression systematically enable us to visualize gene expression profiles in human tumors, and those gene expression profiles can help to identify gene activity pattern that can distinguish subclasses of gastric tumors. Gene profiling studies were also used to better stratify and select patients for adjuvant therapies who may be at higher risk for recurrence. Recently, Gordon G J et al. have shown that simple patterns of gene expression levels, using four to six genes selected from microarray, are highly accurate in the outcome prediction of methothelioma.

However, very few of the previous known studies have collected information from large numbers of genes. Most of them also rely on costly data acquisition platforms, sophisticated algorithms and/or soft wares, and are unable to analyze independent samples without referring to other samples. The clinical applicabilities are therefore limited. In addition, practical methods for identification of individuals with gastric cancer who are at risk for recurrence after surgical resection are not currently available.

### SUMMARY OF THE INVENTION

In order to overcome the drawbacks described in the previous section, a primary object of the present invention is to provide a method for survival prediction after D2 gastrectomy based on gene expression profiles using reverse transcription polymerase chain reaction (RT-PCR) and statistical analysis.

To fulfill the objective of the present invention, a method for determining a survival prediction model for gastric cancer patients after surgical operation using gene expression profiles and RT-PCR comprises the steps of:
(1) obtaining a plurality of pairs of cancerous and noncancerous tissue samples from patients with known survival conditions after surgical operation, performing expression assay of tumor associated genes with a microarray to obtain the gene expression profiles, and selecting special genes expressing significantly differential;
(2) performing RT-PCR analysis of the special genes and confirming the concordance of RT-PCR analysis with the microarray gene expression profile; and
(3) identifying most specific genes among the special genes using a statistical method, and determining a prediction model with the identified most specific genes based on training set samples.

For selecting the special genes with a microarray gene expression profile, for example, step (1) may further comprise but not be limited to the steps of:
(i) normalizing log ratios of expression levels from the expression profiles of each tumor associated genes in the sample tissues;
(ii) filtering out un-significantly expressed genes by fold-change method;
(iii) selecting out the special genes expressing significantly differential using multiple permutation test and cross validation (CV).

The expression levels of the above-mentioned genes could be obtained by the microarray with probes of corresponding cDNA or other corresponding DNA fragments such as oligonucleotides.

In step (2), when comparing the microarray and RT-PCR results of the differentially expressed genes to confirm the consistency, a chosen criteria such as the Spearman rank correlation coefficient with p<0.05 can be selected in the invention.

In step (3), a further logistic regression for selecting tumor-associated genes is preferred. Examples of the above-mentioned statistical method include, but not limited to, stepwise model selection in the invention. To avoid the overfitting problems caused by insufficient sample numbers, the sample number for the training set is preferred to be at least 5-fold of the number of the identified most specific genes in the prediction model.

Another object of the present invention is to provide a method for survival prediction in gastric cancer patients after surgical operation using gene expression profiles. The method can predict survival rates of gastric cancer patients with D2 gastrectomy and provide a reference for following treatments or adjuvant chemotherapy.

The method for survival prediction in gastric cancer patents after surgical operation, comprises:
(a) obtaining pairs of cancerous and noncancerous tissue samples from a patient of gastric cancer;
(b) performing RT-PCR for the identified most specific genes in the samples to detect gene expression levels; and,
(c) predicting the survival of the gastric cancer patient by using the result of RT-PCR from (b) and a survival prediction model determined by the above-mentioned method.

The survival prediction model in the present invention using gene expression profiling techniques is more accurate than the known methods for predicting survival of gastric cancer patients after surgical operation. And methods of the invention realize the clinical availability of microarrays.

### BRIEF DESCRIPTION OF THE DRAWINGS

The related drawings in connection with the detailed description of the present invention to be made later are described briefly as follows, in which:

FIG. 1 (A) illustrates results of reverse transcription PCR for six selected genes and internal control of β-actin; (B) illustrates validation of microarray data for six selected genes using semiquantitative reverse transcription PCR. “N” represents noncancerous tissue; “T” represents cancerous tissue; “CD36” represents CD36 antigen; “SLAM” represents signaling lypmphocytic activation molecule; “TFAP” represents transcription factor AP-2 alpha; “IGF-1” represents insulin-like growth factor 1; “PIM-1” represents PIM-1 insulin-like growth factor 1; “TIMP-4” represents tissue inhibitor of metalloproteinase-4; “G” represents good survival; and “P” represents poor survival.

FIG. 2 illustrates four representative examples of paired RT-PCR status of each selected gene. “CD36” represents CD36 antigen; “SLAM” represents signaling lypmphocytic activation molecule; “TFAP” represents transcription factor AP-2 alpha; “PIM-1” represents PIM-1 insulin-like growth factor 1; “N” represents noncancerous tissue; “T” represents cancerous tissue; “T [symbol] N” represents expression level of cancerous tissue is higher than that of noncancerous tissue; “N [symbol] T” represents expression level of noncancerous tissue is higher than that of cancerous tissue.

FIG. 3 illustrates the survival curves of patients predicted to have good and poor survival. The survival rate of (A) whole testing group; (B) stage III subgroup.

### DETAILED DESCRIPTION OF THE PREFERRED EMBODIMENT

18 patients selected from poor or good survival group were analyzed using an in-house nylon membrane cDNA microarray with colorimetric detection system containing sequences of 328 known genes, and the gene expression profile for predicting survival of gastric cancer patients was screened with a 3-step classification method. First of all, expression levels of 328 genes of cDNA microarrays were obtained from 18 pairs of cancerous and noncancerous gastric tissues and the log ratios of the gene expression levels are determined. The nonlinear LOWESS method, which fits a curve to the log ratios using robust locally weighted regression, was used to normalize the log ratios of the 328 genes to a lowess curve fitting through the MA plot. In the second step, 141 genes out of these 328 genes were extracted using the fold-change method. Finally, the six significantly expressed genes were further extracted using multiple permutation test and cross validation (CV).

The selected genes included CD36 antigen, signaling lypmphocytic activation molecule (SLAM), transcription factor AP-2 alpha (TFAP), insulin-like growth factor 1 (IGF-1), PIM-1 oncogene, and tissue inhibitor of metalloproteinase-4 (TIMP-4).

RT-PCR was performed to test the expression of these six genes in cancerous and noncancerous tissues. The results of RT-PCR were compared with those of microarray. The consistent rates of four out of the six aforementioned genes (CD36, SLAM, TFAP, PIM-1) were high (greater than 60%). And the Spearman's rank correlation coefficients between results of RT-PCR and microarray were significant (p<0.05).

In the invention, four patients out of these 18 patients were randomly chosen for duplicate study to test the reproducibility of the in-house microarrays with nylon membranes. Total RNAs of these four patients were hybridized to two different nylon membrane microarrays at different times. The Pearson's correlation coefficients between hybridizations were all determined to be higher than 0.75 (P<0.05), which represent good reproducibility of this method in the invention.

The expression levels of the 4 selected genes in tumor or non-tumor tissues were classified into 4 categories: (1) Tumor [symbol] Normal, the expression level in tumor is higher than that in normal tissue; (2) Normal [symbol] Tumor, the expression level in normal tissue is higher than that in tumor; (3) the expression levels of Tumor and Normal tissue are both positive, and (4) the expression levels of Tumor and Normal tissue are both negative. Thereafter, RT-PCR expression data from several samples with known survival condition was classified into the above categories. The frequencies of the four categorical RT-PCR result at each predictor in the training group were used to establish the prediction model

The logistic regression together with stepwise model selection was applied to select the prediction model using the Akaike's information criterion (AIC). Three genes (CD36, SLAM, and PIM-1) out of the four above-mentioned genes were extracted to compose the most effective logistic model.

Among these three genes, signaling lymphocytic activation molecule (SLAM) is a CD2-related surface receptor expressed by activated T cells, B cells and dendritic cells. Th0/Th1 immune response, which is usually impaired in gastric cancer patients, could be induced by SLAM to enhance proliferation and cytotoxic ability of CD8+ tumor-specific lymphocytes. Although the exact role of SLAM in tumor-associated immunity of gastric cancer patients remains unclear, it seems to have potential influence on anti-tumor immunity.

CD36 is a trans-membrane receptor that regulates apoptosis and angiogenesis in response to its ligand thrombospondin-1 (TSP-1). TSP-1 is localized to tumor-associated extracellular matrix, and CD36 expressed on surface of tumor cells. The regulation of CD36 expression in tumor cells may play an important role in tumor growth, metastasis and angiogenesis.

PIM-1 is an oncogenic serine/threonine kinase, which can be induced in gastric epithelial cells by Helicobacter pylori infection, and may be involved in gastric carcinogenesis. PIM-1 also plays an important role in proliferation, differentiation and maturation of T-cells, which may be associated with tumor immunity. PIM-1 induced by hypoxia is involved in drug resistance and tumorgenesis of solid tumor cells, which leads to genomic instability. Recently, the expression of PIM-1 has been shown to correlate significantly with measures of clinical outcome in prostate cancer.

Accordingly, the three genes selected in the prediction model of the present invention may be involved in survival associated tumor angiogenesis and tumor immunity.

The prediction model utilizes data from RT-PCR to build classification categories of expression among these three genes in paired samples. Because of its independence of microarray platform after gene selection, the method in the invention requires only small quantities of RNA (as little as 2 μg when performing RT-PCR) and can be easily performed in common laboratory. In the present invention, microarray data normalized with LOWESS method can avoid the systematic error within each microarray sample. The overfitting problem is a crucial issue in the selection process of prediction model. Randomly generated samples were applied to overcome this potential pitfall. And the three-gene prediction model was established which had better sensitivity and specificity than the models with one or two genes.

Adjuvant chemotherapy has been reported to have marginal effect for overall gastric cancer patients with D2 gastrectomy. If survival outcome of gastric cancer patients can be reasonably predicted, adjuvant therapies may help those patients who have high probability of poor survival, while those who have high probability of good survival can be spared for the side effect of adjuvant therapies. Although whether the patients who are predicted to have high probability of poor outcomes really benefit from adjuvant therapies remains unknown, the prediction results could be applied to develop new pharmaceutical compositions in clinical trials or for controlling advanced gastric cancer patients, especially those with stage III disease.

### EXAMPLE 1

### Prediction Model Determination

The 18 gastric tissue samples with cancerous and noncancerous pairs were obtained from 18 patients with gastric cancer who underwent D2 gastrectomy without gross residual tumor at the National Taiwan University Hospital. The tumor stage ranged from stage I to stage IV. Nine patients died of tumor recurrence within 12 months after surgery were defined as ‘poor survival’, and the other nine patients survived beyond 30 months after surgery were defined as ‘good survival’. Poor survival group included two patients with stage II, four with stage III, and three with stage IV. Good survival group included three patients with stage I, two with stage II, and four with stage III. There was no stage I patient in poor survival group, and no stage IV patient in good survival group. All patients did not receive postoperative chemotherapy and radiotherapy. Pair samples of tumor and non-tumor tissues of these 18 patients were dissected and frozen in liquid nitrogen tank within 30 minutes upon removal. Non-tumor mucosa samples were taken from area of grossly normal mucosa located at least 3 cm apart from the tumor border.

Home made nylon membrane microarray which contains 384 spots was applied, which was prepared by the previous known cDNA microarray producing methods in the invention. These 384 spots were aligned by 16 spots in each row and 24 spots in each column with spot diameter of 250 μm. Sequence verified cDNA clones of 328 selected known human genes that are considered to be tumor associated were served as the hybridization targets. These genes include oncogenes, tumor suppressor genes, apoptosis-related genes, matrix proteinase genes, angiogenesis-related genes, immune-related genes, and so on. Internal control genes for microarrays included 16 plant genes and glyceraldehydes phosphate dehydrogenase (GAPDH).

RNAs were extracted from the above-mentioned 18 pairs specimens and used to perform microarray hybridizations. Total RNAs were extracted with Trizol reagent (Invitrogen Life Technologies, Inc. Carlsbad, Calif.). Thirty μg of the total RNAs derived from each gastric cancer tumor tissue and corresponding non-tumor part were reversed transcribed and labeled with biotin.

The microarray membrane carrying the double-stranded cDNAs of tumor associated genes was prehybridized in 1 ml of hybridization buffer (5×RNA extraction standard saline citrate [SSC], 0.1% N-lauroylsarcosine, 0.1% sodium dodecyl sulfate [SDS], 1% blocking reagent mixture manufactured by Roche Molecular Biochemicals, and salmon-sperm DNA [50 μg/mL]) at 63° C. for 1.5 hour The biotin-labeled cDNA probes and hybridization solution (13 μL) containing human COT-1 DNA instead of salmon-sperm DNA were sealed with the microarray in a hybridization bag, and the bag was incubated at 63° C. for 10 hours. The microarray membrane was then washed with 2×SSC containing 0.1% SDS for 5 min at room temperature followed by three washes with 0.1×SSC containing 0.1% SDS at 63° C. for 15 min each. After hybridization, the color reaction was initiated by incubating the membrane for one hour in 1 ml of 1×PBS (phosphate-buffered saline) buffer containing alkaline phosphatase-conjugated streptavidin, 4% polyethylene glycol and 0.3% bovine serum albumin (BSA). The color was developed in BCIP/NBT substrates (5-bromo-4-chloro-3-indolyl-phosphate/nitro blue tetrazolium). Color development was stopped with 1×PBS buffer containing 20 mM EDTA.

After color development, the membrane was scanned with a flat-bed scanner (UMAX [Fremont, Calif.] MagicScan at 3,000 dpi) to get the image. The image was stored in a tagged image file format (Tiff). An image analysis software GenePix Pro software program (Axon Instruments, Foster City, Calif.) was used to quantify the expression levels of the genes.

The color of spots from the enzymatic reaction were converted to gray levels, of which a digital brightness value was assigned to each pixel of one spot ranging from 0 to 256 (from black, through shades of gray, to white). The expression level of each spot in the microarray generated from GenePix Pro 2.0 software after scanning the microarray images with Umax 6000 was collected in an excel file. Expression level of each gene on the microarray was transformed into a log ratio (base 2), which represented expression level of tumor-to-non-tumor tissue.

Data from 18 patients consisting of poor and good survival groups were applied with three-step supervised classification method to extract the most significantly differentially expressed genes between these two survival groups. In the first step, to avoid the systematic error at each microarray sample, the nonlinear LOWESS method which fits a curve to the log ratios using robust locally weighted regression, was used to normalize the log ratios of the 328 genes into a LOWESS curve fitting through the MA plot. Next in the second step, to extract the significantly regulated genes among all 18 microarray samples, the fold-change method which defines the threshold of the significance for expression microarrays was used to define the regulated genes with fold-change (normalized log ratios) in magnitude greater than one at each microarray sample. Among all eighteen microarray samples, the significantly regulated genes with fold-changes in magnitude greater than one for at least two samples among 18 samples were extracted. By this way, 141 genes out of these 328 genes were extracted. In the third step, to extract the most significant differentially expressed genes between these two survival groups, the multiple permutation test was used to test simultaneously all the significantly regulated genes filtered at step two, and the adjusted p value for each gene was obtained. The multiple permutation test is a method to control the probability of producing incorrect test conclusions (false positives and false negatives). To assess the internal consistency of the 18 samples, the leave-one-out cross validation (CV) method was used to generate the 18 CV samples and extracted the differentially expressed genes (whose adjusted p value is less than 0.05 family-wise error rate for all 18 CV samples). Finally, the six significantly expressed genes were further extracted from the aforementioned 141 genes. The six genes are CD36 antigen, signaling lypmphocytic activation molecule (SLAM), transcription factor AP-2 alpha (TFAP), insulin-like growth factor 1 (IGF-1), PIM-1 oncogene, and tissue inhibitor of metalloproteinase-4 (TIMP-4).

To verify the microarray data and to further clarify the difference in the expression of the selected genes, reverse transcription PCR were performed to analyze the selected genes using 10 samples from the 18 samples with sufficient RNA in the microarray study after the genes indicating survival conditions were selected. Two g of total DNA was obtained from reverse transcription using Moloney Murine Leukemia Virus reverse transcriptase, random primers, and other kit reagents (Promega), followed by polymerase chain reaction (PCR). PCR products were separated using electrophoresis on 1.5% agarose gels and visualized under UV light after ethidium bromide staining. The mean band densities were determined using NIH Image 1.62 software, and the levels of selected genes relative to β-actin gene were calculated. The relation between microarray expression ratio and RT-PCR results of six selected genes were determined.

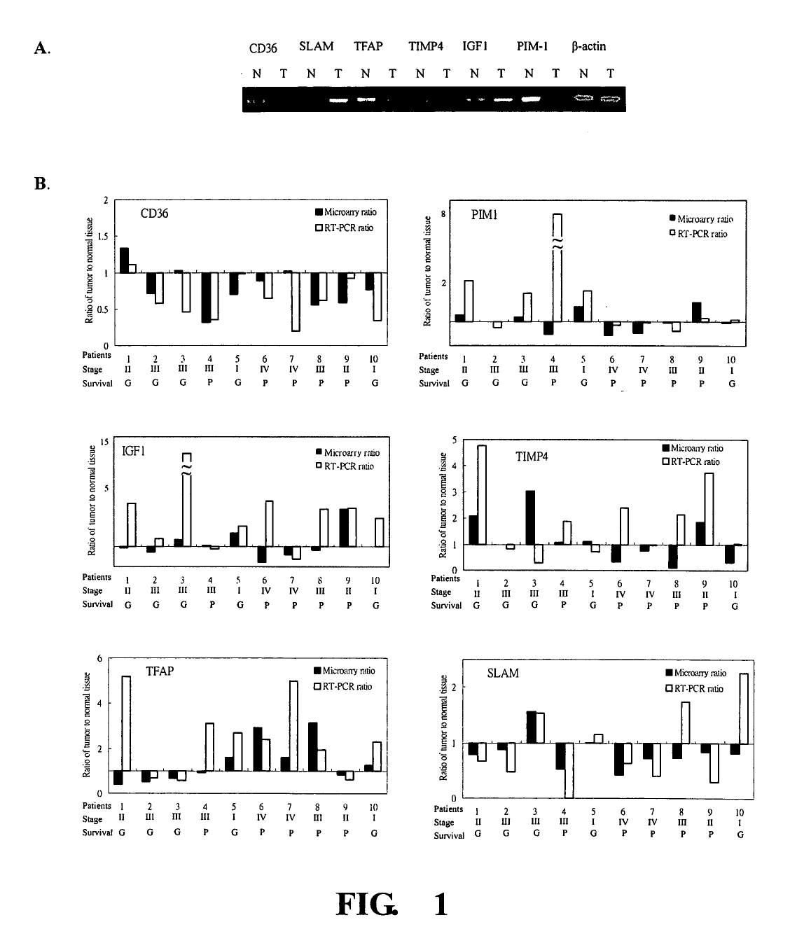

To establish a survival prediction model for gastric cancer patients, the differentially expressed genes whose consistent rates between microarray and RT-PCR results were greater than 60% or Spearman rank correlation coefficient showed significant and p<0.05 were selected for prediction model training (FIG. 1). Four genes were selected thereafter, that are CD36, SLAM, TFAP and PIM-1.

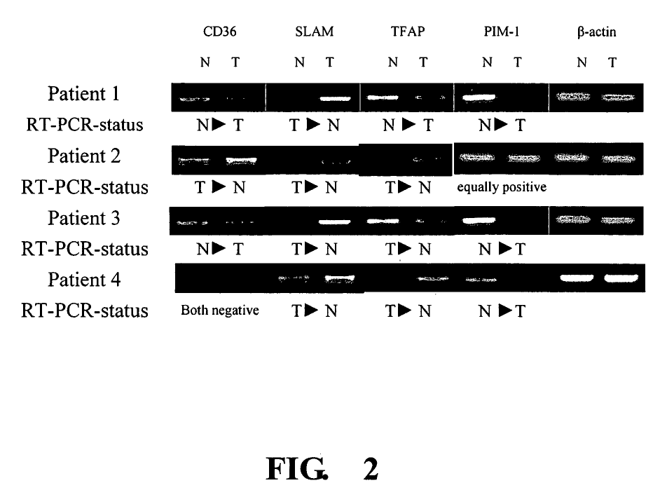

The RT-PCR expression levels of the selected genes in tumor or non-tumor tissues were classified into 4 categories: (1) Tumor [symbol] Normal, the expression level in tumor is higher than that in normal tissue; (2) Normal [symbol] Tumor, the expression level in normal tissue is higher than that in tumor; (3) the expression levels of Tumor and Normal tissue are both positive, and (4) the expression levels of Tumor and Normal tissue are both negative (FIG. 2). Thereafter, 10 samples from the aforementioned 18 samples and 10 samples selected randomly from another 40 newly enrolled patients were served as the training group of 20 samples for the prediction model. Among them, 10 samples are in good survival group and 10 are in poor survival group. The RT-PCR status of these samples was classified into the 4 categories. The frequencies of the 4 categorical RT-PCR results at each predictor in the training group were used to establish the prediction model. And then, the logistic regression together with stepwise model selection was applied to select the effective prediction model using the Akaike's information criterion (AIC). Three genes (CD36, SLAM, and PIM-1) out of the four above-mentioned genes were extracted to compose the most effective logistic model. The prediction formula is represented by Formula 1:
λ=0.833 CD36−0.762 SLAM−0.317 PIM-1
π=exp(λ)/(1+exp(λ)) (Formula 1)
wherein, CD36, SLAM, and PIM-1 represent the corresponding frequencies of CD36, SLAM, and PIM-1 respectively in the above-mentioned RT-PCR categories; π is the probability of “poor survival status”.

Good survival (defined as survival time >30 months) was predicted when π is less than or equals to 0.5. Poor survival (defined as survival time <12 months) was predicted when π is greater than 0.5. The standard errors of the logistic regression coefficients are 0.411 for CD36, 0.436 for SLAM, and 0.173 for PIM-1 respectively.

People who skilled in the art will easily understand through reading the above-mentioned description of the instruction, the coefficients in Formula 1 listed may vary a little according to the difference of patients or the number of samples in the training group, which will not affect the invention to practice. It is understandable that the more samples included in the training group for prediction model, the more accurate the prediction formula is.

### EXAMPLE 2

### Prediction Model Testing

The survival prediction model consisting of three genes (CD36, SLAM, PIM-1) developed in the invention was applied in a 30 newly enrolled patients as an independent test group to predict the survival condition. RT-PCR was carried out with the tumor and non-tumor samples from 30 patients to analyze the expression profiles of CD36, SLAM, PIM-1. The RT-PCR statuses of genes were translated into the categorical variables as mentioned in Example 1 to get the frequencies of the 20 genes in the training group. The corresponding frequencies of each gene in each patient were entered into Formula 1 to obtain the survival prediction of the gastric cancer patient after gastrectomy.

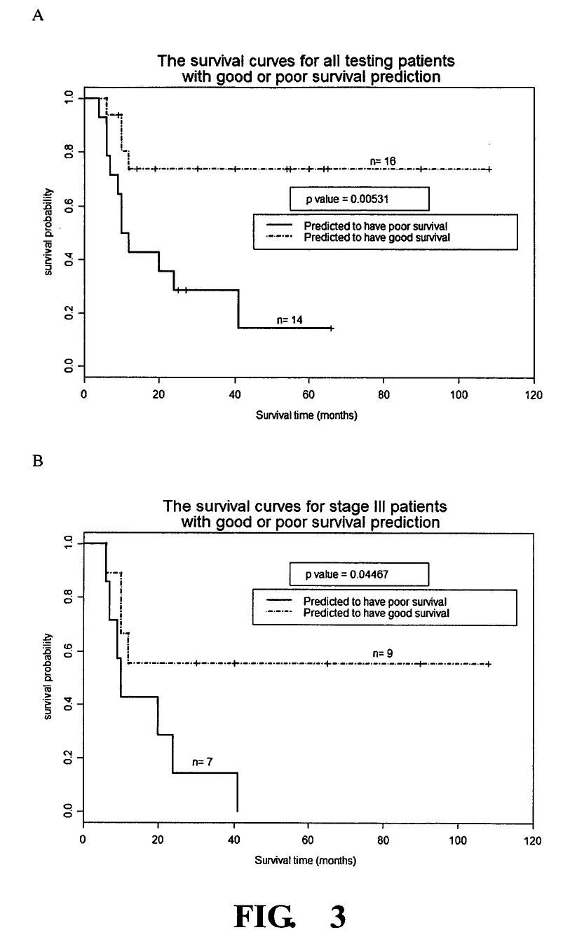

Survival of twenty-three patients (76.7%) were correctly predicted, and yielded a specificity of 80%, a sensitivity of 73.3%, a positive prediction value of 75%, and a negative prediction value of 78.57%. The frequency distribution was showed in Table 1A. This reveals that this prediction model showed highly predictive power in the independent test group. The survival rate of the patients predicted to have good survival was significantly higher than that of the patients predicted to have poor survival (p=0.00531) (FIG. 3A).

Of the seven stage I patients, six were correctly predicted by this model. One patient was predicted as poor survival, and died of multiple liver metastases in 12 months. Of the other 6 patients, five was correctly predicted, and the frequency distribution was showed in Table 1B. Of the five stage II patients, three were correctly predicted. Two of three patients predicted to have poor survival died of disease in 12 months, and the frequency distribution was showed in Table 1C. Two stage IV patients were correctly predicted by this model, and the frequency distribution was showed in Table 1E.

The prediction model was applied to 16 patients with stage III disease, and the frequency distribution of accuracy was showed in Table 1D. Twelve patients (75%) were correctly predicted, and yielded a specificity of 100%, a sensitivity of 63.6%, a positive prediction value of 100%, and a negative prediction value of 55.6%. The survival rate of the patients predicted to have good survival was significantly higher than that of patients predicted to have poor survival (p=0.04467) (FIG. 3B).

TABLE 1A
Frequency distribution of accuracy in the whole 30 test patients

| | | Clinical survival status | | |
|---|---|---|---|---|
| | | Poor | Good | Total |
| Predicted survival status | Poor | 11 | 3 | 14 |
| | Good | 4 | 12 | 16 |
| | Total | 15 | 15 | 30 |

Sensitivity = 73.33%
Specificity = 80.00%
Negative predictive value = 78.57%
Positive predictive value = 75.00%

TABLE 1B
Frequency distribution of accuracy in the seven stage I patients

| | | Clinical survival status | | |
|---|---|---|---|---|
| | | Poor | Good | Total |
| Predicted survival status | Poor | 1 | 1 | 2 |
| | Good | 0 | 5 | 5 |
| | Total | 1 | 6 | 7 |

Sensitivity = 100.00%
Specificity = 85.71%
Negative predictive value = 100.00%
Positive predictive value = 50.00%

TABLE 1C
Frequency distribution of accuracy in the five Stage II patients

| | | Clinical survival status | | |
|---|---|---|---|---|
| | | Poor | Good | Total |
| Predicted survival status | Poor | 2 | 1 | 3 |
| | Good | 1 | 1 | 2 |
| | Total | 3 | 2 | 5 |

Sensitivity = 50.00%
Specificity = 66.67%
Negative predictive value = 66.67%
Positive predictive value = 50.00%

TABLE 1D
Frequency distribution of accuracy in the 16 Stage III patients

| | | Clinical survival status | | |
|---|---|---|---|---|
| | | Poor | Good | Total |
| Predicted survival status | Poor | 7 | 0 | 7 |
| | Good | 4 | 5 | 9 |
| | Total | 11 | 5 | 16 |

Sensitivity = 63.64%
Specificity = 100.00%
Negative predictive value = 55.56%
Positive predictive value = 100.00%

TABLE 1B
Frequency distribution of accuracy in the two stage IV patients

| | | Clinical survival status | | |
|---|---|---|---|---|
| | | Poor | Good | Total |
| Predicted survival status | Poor | 1 | 0 | 1 |
| | Good | 0 | 1 | 1 |
| | Total | 1 | 1 | 2 |

Sensitivity = 100.00%
Specificity = 100.00%
Negative predictive value = 100.00%
Positive predictive value = 100.00%

What is claimed is:

1. A method for determining a survival prediction model for gastric cancer patients after surgical operation using gene expression profiles and RT-PCR, comprises the steps of:
(1) obtaining a plurality of pairs of cancerous and noncancerous tissue samples from patients with known survival conditions after surgical operation, performing expression assay of tumor associated genes with a microarray to obtain the gene expression profiles, and selecting special genes expressing significantly differential;
(2) performing RT-PCR analysis of the special genes and confirming the concordance of RT-PCR analysis with the microarray gene expression profile; and
(3) identifying most specific genes among the special genes using a statistical method, and determining a prediction model with the identified most specific genes based on training set samples.

2. The method as claimed in claim 1, wherein the tumor associated genes in step (1) comprise at least one of the following genes: oncogenes, tumor suppressor genes, apoptosis-related genes, matrix proteinase genes, angiogenesis-related genes, and immune-related genes.

3. The method as claimed in claim 1, wherein in step (1) further comprises the steps of:
(i) normalizing log ratios of expression levels from the expression profiles of each tumor associated gene in the sample tissues;
(ii) filtering out un-significantly expressed genes by fold-change method;
(iii) selecting out the special genes expressing significantly differential using multiple permutation test and cross validation (CV).

4. The method as claimed in claim 3, wherein the microarray is a DNA microarray.

5. The method as claimed in claim 3, wherein step (i) is performed with nonlinear locally weighted regression.

6. The method as claimed in claim 1, wherein the concordance in step (2) is confirmed with a chosen criterion.

7. The method as claimed in claim 6, wherein the chosen criterion is a Spearman rank correlation coefficient with p<0.05.

8. The method as claimed in claim 1, wherein the statistical method is a stepwise model selection.

9. The method as claimed in claim 1, wherein step (3) further comprises selecting tumor-associated genes by logistic regression.

10. The method as claimed in claim 1, wherein the training set samples in step (3) are from a plurality of pairs of cancerous and noncancerous tissue samples with known survival conditions after surgical operation.

11. The method as claimed in claim 10, wherein the number of training set samples is not less than 5 times of the number of the identified most specific genes in the prediction model.

12. The method as claimed in claim 1, wherein the special genes comprise at least one of the following genes: CD36 antigen, signaling lypmphocytic activation molecule (SLAM), transcription factor AP-2 alpha (TFAP), insulin-like growth factor 1 (IGF-1), PIM-1 oncogene, and tissue inhibitor of metalloproteinase-4 (TIMP-4).

13. The method as claimed in claim 1, wherein the identified most specific genes are selected from the group consisting of CD36 antigen, signaling lypmphocytic activation molecule (SLAM), transcription factor AP-2 alpha (TFAP), and PIM-1 oncogene.

14. The method as claimed in claim 1, wherein the identified most specific genes are selected from the group consisting of CD36 antigen, signaling lypmphocytic activation molecule (SLAM), and PIM-1 oncogene.

15. A method for survival prediction in gastric cancer patents after surgical operation, comprises:
(a) obtaining pairs of cancerous and noncancerous tissue samples from a patient of gastric cancer;
(b) performing RT-PCR for a plurality of identified most specific genes in the samples to detect gene expression levels; and
(c) predicting the survival of the gastric cancer patient by using the result of RT-PCR from (b) and a survival prediction model determined by the method as claimed in claim 1.

16. The method as claimed in claim 15, wherein the noncancerous tissue samples were taken from an area located no less than 3 cm apart from the cancerous tissue.

17. The method as claimed in claim 15, wherein the identified most specific genes comprise at least one of the following genes: CD36 antigen, signaling lypmphocytic activation molecule (SLAM), transcription factor AP-2 alpha (TFAP), insulin-like growth factor 1 (IGF-1), PIM-1 oncogene, and tissue inhibitor of metalloproteinase-4 (TIMP-4).

18. The method as claimed in claim 15, wherein the identified most specific genes are selected from a group consisting of CD36 antigen, signaling lypmphocytic activation molecule (SLAM), and PIM-1 oncogene.

19. The method as claimed in claim 18, wherein the survival prediction model is a formula as Formula 1 of:
λ=0.833 CD36−0.762 SLAM−0.317 PIM-1
π=exp(λ)/(1+exp(λ)) (Formula 1)
wherein, CD36, SLAM, and PIM-1 represent the corresponding frequencies of CD36, SLAM, and PIM-1 respectively in training set samples in the RT-PCR categories including the expression level in cancerous tissue is higher than that in noncancerous tissue; the expression level in noncancerous tissue is higher than that in cancerous; the expression levels of cancerous and noncancerous tissue are both positive, and the expression levels of cancerous and noncancerous tissue are both negative; π is a probability of a “poor survival status”, good survival is predicted when π is less than or equals to 0.5, and poor survival is predicted when π is greater than 0.5.

20. The method as claimed in claim 19, wherein the good survival is defined when survival time is no less than 30 months.

21. The method as claimed in 19, wherein the poor survival is defined when survival time is no more than 12 months.
